(19) United States
(12) Patent Application Publication
(10) Pub. No.: US 2025/0182850 A1
(43) Pub. Date: Jun. 5, 2025

(54) CREATION OR USE OF ANCHOR-BASED DATA STRUCTURES FOR SAMPLE-DERIVED CHARACTERISTIC DETERMINATION

(71) Applicant: COSMOSID INC., Rockville, MD (US)

(72) Inventors: Nur A. HASAN, College Park, MD (US); Tom CEBULA, Baltimore, MD (US); Boyd Thomas LIVINGSTON, Columbia, MD (US); Huai LI, Clarksville, MD (US); David JAKUPCIAK, Bethesda, MD (US); Rita R. COLWELL, Bethesda, MD (US); Douglas M. BRENNER, Colleyville, TX (US)

(21) Appl. No.: 19/047,193

(22) Filed: Feb. 6, 2025

Related U.S. Application Data
(63) Continuation of application No. 16/853,712, filed on Apr. 20, 2020.
Application No. 16/853,712 is a continuation of application No. 13/836,139, filed on Mar. 15, 2013, now abandoned.

(51) Int. Cl. G16B 30/00; G16B 20/00; G16B 20/20; G16B 30/20; G16B 40/00; G16B 40/10

(52) U.S. Cl. CPC G16B 30/00; G16B 20/20; G16B 30/20; G16B 40/00; G16B 40/10; G16B 20/00

(57) ABSTRACT

In some embodiments, sample-derived characteristic determination may be facilitated via creation or use of anchor-based data structures. In some embodiments, an anchor and a seed length range may be obtained (e.g., for creating a reference data structure derived from reference data). Based on the anchor and the seed length range, reference seeds may be extracted from the reference data (e.g., such that each of the extracted reference seeds (i) is a data instance adjacent at least one instance of the anchor in the reference data and (ii) has a length within the seed length range). The reference data structure may be created with the extracted reference seeds, and unassembled sample data may be processed using the reference data structure, the anchor, and the seed length range to determine characteristics related to the unassembled sample data.

### CROSS-REFERENCE TO RELATED APPLICATIONS

This application is a continuation of U.S. patent application Ser. No. 16/853,712, filed Apr. 20, 2020, which claims priority to U.S. patent application Ser. No. 13/836,139, filed Mar. 15, 2013, the entire content of all applications are incorporated herein by reference in their entireties.

### FIELD OF THE INVENTION

This invention relates to a system, apparatus and methods for the characterization of biological material in a sample, and, more particularly, to the characterization of the identities and/or traits of biological material in a sample and/or the relative abundances of the identified biological material or traits thereof.

### BACKGROUND OF THE INVENTION

Accurate and definitive microorganism identification, including microbial identification and pathogen detection, is essential for accurate disease diagnosis, treatment of infection and trace-back of disease outbreaks associated with microbial infections. Microbial identification is used in a wide variety of applications including medical diagnosis, food safety, drinking water, microbial forensics, criminal investigations, bio-terrorism threats and environmental studies. It is crucial for effective disease control but also as an early warning system for emergence of epidemics and attacks using microbiological agents as weapons. Advances in nucleic acid (NA) sequencing technologies have made it possible for scientists to sequence complete microbial genomes rapidly and efficiently. Access to the NA sequences of entire microbial genomes offers a unique opportunity to analyze and understand microorganisms at the molecular level and to design novel approaches for microbial pathogen detection and drug development. Identification of microbial pathogens as etiologic agents responsible for chronic diseases is leading to new treatments and prevention strategies for these diseases.

Antony van Leeuwenhoek (1632-1723) developed techniques for improving lens magnification to the point where he was able to see and describe “strange little animals,” which he could not have possibly known would in the future demonstrate the ability to harm cells, agricultural crops, animals, and human bodies. Leeuwenhoek's discoveries were some of the first recorded biological agent detection methods on record, although it was not until Louis Pasteur and Robert Koch established that these bacteria could cause diseases that the hunt was on for biological agents.

Although microscopy was the first method to identify bacteria, other classes of biological agent detection methods have also been developed with both advantages and disadvantages over microscopy, including bioassays, antibody-based approaches, Polymerase Chain Reaction (PCR) methods, DNA microarray, sequencing, in situ hybridization, and mass spectrometry.

a. Conventional Culture

Classical methods for detecting and identifying microorganisms require isolating the organisms in pure cultures, followed by testing for multiple physiological and biological traits. Established methods, relying on culturing for identification include an evaluation of the microorganism's ability to grow in media exposed to multiple conditions. The general method of detection by culture can be broken down into the following steps: general enrichment, selective enrichment, bioassay screening and confirmation. A key drawback to detecting and identifying infectious agents by culturing, and subsequent bioassays that rely on culturing, is the inability of the target organism to grow in adequate amounts.

Of the microorganisms that can be cultured, a further drawback is that identification can be compromised by overgrowth of competitor microorganisms in the sample, thus masking the target microorganism. Exotic or uncommon pathogens are particularly hard to identify this way.

Finally, a most serious drawback to culture in the clinical diagnostic environment is that the culturing process can take several days. Treatment decisions, including, for example, choice of an effective antibiotic in the case of infection, will be delayed until the microorganism is cultured in isolation.

b. Serology/Immunoassay/Antibody Assay

Currently the most widely utilized method for bacterial and virus detection in clinical microbiology and laboratory diagnostics is the serological test, which has many forms and uses for detecting and identifying single isolates. Only recently, however, have many FDA-approved kits for the detection of a single bacterium or virus become commercially available. As recently as 1999, a review of the published literature showed that only a few antigen-based detection methods were commercially available. A little more than a decade later immunological testing has become the dominant detection method for single isolate detection and identification. The reasons for lack of commercial use previously were the challenges in creating assays that were both reliable and effective in routine applications.

One complication was the fact that classic strategies for immunoreactive antibody production relied on the use of the entire bacterium or identification and testing of proteins selected empirically. These obstacles were overcome by the introduction of monoclonal antibodies and techniques used to target antigens and discover new unique peptides for biological agents such as the MALDI-TOF mass spectrometry. Other advances include the advancement in the quality and specificity of reagents and development of reference laboratories to which researchers submit cell-culture isolates for serological production. Although immunoassay-based tests are rapid, a key drawback is the lack of specificity, due to the fact that antibodies produced against one antigen can often cross-react with other antigens, leading to false positive identifications compounded by the high sensitivity of immunoassays. In addition, the reliability of this method can be severely compromised by a false negative antigen-antibody reaction caused by an excessive amount of antibody, or excess antigen resulting in no lattice formation in an agglutination reaction.

c. Microscopy

There are several different types of microscopy techniques ranging from direct epifluorescence filter technique (DEFT), flow cytometry, direct fluorescence antibody techniques, and electron microscopy. Microscopy detection methods utilize direct observation for detection, and early microscopy that utilized light had a minimum detection range of around 250 nm. Major improvements to microscopy include combination with fluorescence antibody techniques and electron microscopy and, more recently, the introduction of computerized automated microscopy. To further improve automation, instead of samples applied or fixed to a slide, the sample can be run through a flow cytometer connected to the microscopy equipment, thereby automating the system even more. Other problems with visualization of the biological target were overcome through the development of enrichment and/or filtration steps before application of the probes. With the addition of automation, fluorescence probes, and computer visualization, microscopy can now classify individual bacterial cells within a mixed population.

Drawbacks to most microscopic methods include the requirement first to culture the microorganism, the high level of expertise needed to conduct microscopic analyses, and the expense of microscopy equipment.

d. Mass Spectrometry

There are several types of mass spectrometers, such as gas and liquid chromatography mass spectrometry, and matrix-assisted laser desorption ionization-time of flight (MALDI-TOF) mass spectrometry. Every mass spectrometer consists of three fundamental components: an ion source; a mass analyzer; and a detection device. Current methods utilizing mass spectrometers focus on either the detection of proteins and peptides or the detection of nucleic acids. The most advanced methods of mass spectrometry detection have recently reported 86.8% identification ability compared to conventional procedures, with slightly lower capabilities when identifying streptococcal species. A major improvement to mass spectrometry is the capability to apply the method directly to crude samples yet still obtain data having a quality high enough to allow for classification. Additionally, mass spectrometry has the ability to identify post-translational modifications. The most important development in the field of mass spectrometry is the improved ability to automate the system the enhanced computational analysis techniques.

Because this method analyzes only the protein mass profile, and no other protein analysis is done, it is not an efficient way to identify antibiotic resistant or virulent factors. Another difficulty is that the sample may need to be cultured in order to get enough material to analyze. Likewise, low protein mass organisms such as viruses are not good candidates for this method. Lastly, this method works best with cultured isolates; it is not meant for metagenomic samples.

e. Polymerase Chain Reaction

Polymerase Chain Reaction (PCR) represents one of the simplest approaches to detection of biological agents. PCR has several variations, including real-time PCR, reverse-transcription (RT) PCR, targeted PCR, and random PCR; thus lending the method for extensive use in detection of biological agents and determination of actual disease detection. In all PCR methods, there are several basic components: a target sequence that can either be DNA or ribonucleic acid (RNA), amplification primers that can be either targeted or random depending on the method, detection of the amplification product that can be fluorescence based, sequencing based, or hybridization based. One improvement offered with PCR-based methods over traditional diagnostic tests is that organisms do not require culturing before detection. PCR is highly sensitive, and it can be very selective and rapid. PCR is often utilized in other detection methods that are DNA based, as it is highly selective and requires small quantities of starting material.

Since PCR-based methods rely on primer-specific amplification of genetic material, they necessarily require advanced knowledge of the genome sequence of the target organism to design successful assays. Furthermore, the high specificity of the method prevents detection of microorganisms that have mutations in the primer region.

f. Microarray

Developed since the middle of the last decade, microarrays represent the evolution of traditional membrane-based blots, where a labeled probe hybridizes to a target. The difference is that, in membrane-based methods, the sample DNA is attached to the substratum and probes are hybridized to it, whereas, in array-based methods, the probes are bound to the substratum and sample DNA hybridized to the targeted probes. Hybridization based approaches, such as microarray-probes, require known or predicted answers for detection of biological treats. With microarrays the probe targets can be proteins or nucleic acid based. Field based applications of microarrays have been used successfully for the detection of biological agents like V. cholerae and other organisms. Since microarrays can scan large amounts of data for several different organisms, the technology lends itself to uncovering important underlying factors associated with infection and other relationships. DNA and RNA based hybridization using microarrays originally did not have the desired sensitivity, but combining the microarray technology with PCR based technologies have drastically improved the sensitivity.

g. Detection of Multiple Microorganisms in Mixed Samples

Methods for identifying a single microorganism in a sample have become valuable tools in the diagnostic field; however, it can be advantageous to detect and identify multiple microorganisms in a single sample with a broader level test. The most common methods for such identification are: denaturing gradient gel electrophoresis (DGGE), DNA microarrays (described above), 16S gene sequencing, and metagenomic sequencing. A common advancement with all of these technologies is their ability to utilize products of PCR, thus making the methods very selective and sensitive.

### g1. Denaturing Gradient Gel Electrophoresis (DGGE)

DGGE is a method that allows for the detection and identification of microbial populations in addition to single isolates. In DGGE, target sequences are amplified by PCR using primers targeted to the 16s ribosomal gene, and PCR amplicons are separated using electrophoresis in a denaturing gradient. Some have used the banding pattern in the gel to determine the composition of the microbial community in the sample. Ultimately, for the identification of the metagenomic community the bands of amplified DNA are cut out from the gels for sequencing and further phylogenetic analysis.

A serious drawback in DGGE analysis of metagenomic samples is the use of universal primers that fail to amplify in cases where there are mismatches between the binding site on the genome and the primers. An advancement in the technology has been the introduction of software for gel analysis. Another major drawback with the DGGE technique is its failure to effectively utilize PCR products larger than 600 bp. Another disadvantage is the failure to resolve multiple genes when multiple gene complexes are amplified in a single PCR reaction; furthermore, if any preferential amplification occurs, then the detection and identification of all the genes is compromised. Other significant problems are heteroduplex and the co-migration of distinct sequences. Therefore, without sequencing, issues such as heteroduplex, preferential amplification, and co-migration can confuse any interpretations of DGGE results. Also a significant amount of optimization is required before maximal separation of various sequences is achieved on a reliable basis, and even slight variations in concentration of the denaturants or gel reagents can result in unexpected results.

### g2. Microarray

For metagenomic detection, microarrays have several probes for a range of targets; thus, broadening the number of detectable organisms. The probes can either be protein or nucleic acid based. Improvements such as microarray printing allow microarrays to achieve high-throughput rates by sampling thousands of test samples with a single test. However, certain probes do not always function effectively using the microarray method; thus, the probes will not yield the expected signals in the presence of the targeted organisms and the microarray designers must account for false negatives before the test enters into production. Additionally, different probes do not always have the same target-binding capacities, causing difficulties when interpreting microarray results. Problems, such as image analysis of the data and creating optimal detection rules allowing accurate identification of all the biological agents create challenges that must be reconciled before the introduction of microarray chips. However, the major issue always revolves around hybridized based approaches that can only detect information on predicted/predetermined answers and are often unreliable from experiment to experiment. With regard to protein based antibodies, the selected antigen may have been expressed only under specific exposure events; therefore, when that event does not occur, the biological agent may become undetectable.

g3. 16S rRNA Gene Sequencing

16S rRNA gene sequencing has enhanced the taxonomical classification of bacteria by creating a method to trace phylogenetic relationships between and among organisms. The ribosomal RNA gene contains regions with variable degrees of nucleotide diversity, ranging from highly conserved to extremely variable. Additionally, numerous bacterial 16S rRNA genes have been sequenced and are publicly available, creating a large library for comparison. Overall, relationships of 16S rRNA genes below 97% sequence identity when comparing two sequences are indicative of different species. Selective amplification of the 16S rRNA genes can allow for a very sensitive method; therefore, multiple methods utilize the 16S rRNA region, such as, DGGE, microarray, and sequencing.

By selectively amplifying using PCR, the 16S rRNA gene fragments allow the investigator to identify multiple organisms in mixed samples. In some sample types though, the 16S rRNA gene can give a weak signal compared to other probes. One drawback of the 16S rRNA technique is that, when mutation occurs in the sequences of the primer binding site, false negatives arise and can result in the inability to identify particular bacteria. Some organisms express variable sequences in regions with expected conserved domains; therefore, identification employing amplification of the 16S rRNA and using universal primers becomes difficult. Furthermore, 16S rRNA may not permit identification at the species level since the 16S rRNA sequence is highly conserved within some genera. A major drawback with 16S rRNA sequencing is false signals due to background DNA and how to reduce the noise generated from high concentration organisms.

16s rRNA gene sequencing is not robust at the species level. The method cannot always identify strains that are antibiotic resistant or virulent. Furthermore, for metagenomic identification, the presence of large genomic backgrounds is likely to reduce the specificity and detection resolution of the test. Finally, the method requires a cultured sample in order to have enough material to run the assay. It is now well understood that a single gene may not be adequate to yield an accurate identification to the species or subspecies level and additional gene sequences along with other data may be required. Confounding issues include non-uniform distribution of sequence dissimilarity among different taxa and instances in which multiple copies of the 16S rRNA gene may be present in the same organism that differ by more than 5% sequence dissimilarity. This can lead to different presumptive identifications for the same individual, depending on which 16S rRNA gene is analyzed.

### g4. Metagenomic Sequencing and Assembly of Microbial Genomes

Assembly of the full microbial sequence is tedious, error prone at present, and unlikely to be automated and error free in the near future. Furthermore attaining the full sequence of all microorganisms in a metagenomic sample on a quantitative basis is unattainable by present technology. Identification of such a massive data set would require access to massive computing capability and requires culturing to obtain the individual component strains.

The problem of species identification in a mixture of organisms has been evidenced in the case of certain static marker-based metagenomic methods, such as the ribosomal genes (16S, 18S, and 23S rRNA) or coding sequences of genes involved in the transcription or translation machinery of the cell (e.g., recA/radA, hsp70, EF-Tu, Ef-G, rpoB). By definition, such markers are based on slow-evolving genes. The aim of the marker-based metagenomic methods is to distinguish between species with large evolutionary distances, and, thus, it is unsuitable for resolving closely related organisms. Although microbial 16S rDNA sequencing is considered the gold standard for characterization of microbial communities, it may not be sufficiently sensitive for comprehensive microbiome studies. rRNA gene-based sequencing can detect the predominant members of the community, but these approaches may not detect the rare members of a community with divergent target sequences. Primer bias and the low depth of sampling account for some of the limitations microbial 16S rDNA sequencing, which could be improved with sequencing of entire microbial genomes.

To overcome the limitations of single gene-based amplicon sequencing by pyrosequencing, whole-genome shotgun sequencing has emerged as an attractive strategy for assessing complex microbial diversity in mixed populations. Whole genome-based approaches offer the promise of more comprehensive coverage by high-throughput, parallel DNA-sequencing platforms, because they are not limited by sequence conservation or primer-binding site variation within a specific target. Fueled by the innovations in high-throughput DNA sequencing, the rate of genomic discovery has grown exponentially with the increasing need for high-performance computing and bioinformatics. The primary challenge for such whole genome based approach is how to obtain accurate microbial identification for hundreds or thousands of species in a reasonable time and for a reasonable cost.

Current bioinformatics throughput is too slow and not sufficiently automated for large-scale projects, and often requires trimming, assembly, alignments and annotations. Even then, sufficient computational power like distributed computing networks and robust server technology, time and manpower appear to be crucial. Once high-quality sequences have been obtained from mixed species communities, the next challenge is to accurately identify many microbes in parallel. Current bioinformatics pipelines available today like BLAST, BLASTZ, netBlast, BlastX-MEGAN, MG-RAST, IMG/M, short read mapping and other comparison tools can only allow for a rough identification of a microbial community of interest and cannot distinguish between discrete species and populations of closely related biotypes. While these tools create alignments of variable length from sequence intervals of unspecified phylogenetic relevance, potential problems of false positives may appear. Assignments based on very short read (<50 bp) usually suffer from low confidence values, whereas reads of length ˜100 bp may be assigned with a reasonable level of confidence (BLASTX bit-scores of 30 and higher) can identify only at species level and result in severe under-prediction. Finally, rapid development of current “next-generation” sequencing (NGS) technologies indicates that the future genome-based technologies will be “smaller, cheaper, and faster.” This warrants the need for a quick and sophisticated bioinformatics tools to identify a genetic resource, with a high degree of accuracy and reliability, at the point of need, and at the ease of computation and time.

### SUMMARY

In some embodiments, sample-derived characteristic determination may be facilitated via creation or use of anchor-based data structures. In some embodiments, an anchor and a seed length range may be obtained (e.g., for creating a reference data structure derived from reference data). Based on the anchor and the seed length range, reference seeds may be extracted from the reference data (e.g., such that each of the extracted reference seeds (i) is a data instance adjacent at least one instance of the anchor in the reference data and (ii) has a length within the seed length range). The reference data structure may be created with the extracted reference seeds, and unassembled sample data may be processed using the reference data structure, the anchor, and the seed length range to determine characteristics related to the unassembled sample data.

As discussed herein, pre-existing whole genome-based methods require high-performance computing and bioinformatics, thereby failing to obtain accurate microbial identification for hundreds or thousands of species in a reasonable time and for a reasonable cost. This is because assembly of the full microbial sequence is tedious, error prone at present, and unlikely to be automated and error free in the near future, and identification of such a massive data would require access to massive computing capability. Such whole genome-based methods also generally can identify only at species level which result in severe under-prediction. In other words, despite having the computational power of distributed computing networks and robust server technology, pre-existing whole genome-based methods and its current bioinformatics throughput are too slow and often fails to accurately identify beyond the species level (e.g., they fail to accurately identify at the sub-species or strain level). Through the use of anchors and short seeds, such embodiments described herein can distinguish pathogens or other microorganisms even between different strains present in metagenomic data in just a few minutes. With respect to such embodiments, the need to first assemble the fragment data into contiguous segments (contigs) or whole genomes may be avoided.

### BRIEF DESCRIPTION OF THE DRAWINGS

The accompanying drawings, which are incorporated herein and form part of the specification, illustrate various embodiments of the present invention. In the drawings, like reference numbers indicate identical or functionally similar elements.

FIG. 1 is a schematic illustration of an instrument capable of characterizing biological material in a sample or isolate according to an embodiment of the present invention.

FIG. 2 is a schematic illustration of an instrument capable of characterizing biological material in a sample or isolate according to an embodiment of the present invention.

FIG. 3 is flowchart illustrating a process that may be performed to characterize biological material in a sample or isolate according to an embodiment of the present invention.

FIG. 4 is flowchart illustrating a process that may be performed to characterize biological material in a sample or isolate according to an embodiment of the present invention.

FIG. 5 is a flowchart illustrating a first comparator engine that may be used to characterize biological material in a sample or isolate according to an embodiment of the present invention.

FIG. 6 is a flowchart illustrating a second comparator engine that may be used to characterize biological material in a sample or isolate according to an embodiment of the present invention.

### DETAILED DESCRIPTION

In the following description, for the purposes of explanation, numerous specific details are set forth in order to provide a thorough understanding of the embodiments of the invention. It will be appreciated, however, by those having skill in the art that the embodiments of the invention may be practiced without these specific details or with an equivalent arrangement. In other cases, well-known structures and devices are shown in block diagram form in order to avoid unnecessarily obscuring the embodiments of the invention.

This present invention relates to a system, apparatus and methods for the characterization of biological material in a sample, and, more particularly, to the characterization of the identities and/or traits of biological material in a sample and/or the relative abundances of the identified biological material or traits thereof. The characterization may rely on probabilistic methods that compare sequencing information of fragment reads to sequencing information of reference genomic databases and/or trait-specific database catalogs.

In one aspect, the present invention provides a method of characterizing organisms based on sequence information derived from a sample containing genetic material from the organisms. The method may include (a) receiving, by a processing unit including a processor and memory, the sequence information derived from the sample. The sequence information may include unassembled nucleotide fragment reads. The method may include (b) performing, by the processing unit, probabilistic methods that compare the unassembled nucleotide fragment reads with trait-specific reference sequence information contained in a trait-specific database catalog and produce probabilistic trait results. The method may include determining, by the processing unit, one or more traits associated with the organisms using the probabilistic trait results.

In some embodiments, the method may include: (d) performing, by the processing unit, probabilistic methods that compare the unassembled nucleotide fragment reads with reference sequence information contained in a reference database containing genomic identities of organisms and produce probabilistic identity results; and (e) determining, by the processing unit, the identities of the organisms contained in the sample at least at the species level using the probabilistic identity results.

In some embodiments, the reference sequence information contained in the reference database may be assembled or partially assembled sequence information. The organisms may be microorganisms, and the reference database may comprise a microbial whole genome database. The method may include determining, by the processing unit, the identities of the organisms contained in the sample at the species or sub-species levels using the probabilistic identity results. The method may include determining, by the processing unit, the identities of the organisms contained in the sample at the strain level using the probabilistic identity results.

In some embodiments, steps (d) and (e) may be performed while steps (b) and (c) are performed. In other embodiments, steps (b) and (c) are performed after steps (d) and (e) have been performed.

In some embodiments, the method may include characterizing the relative populations or abundance of species and/or sub-species and/or strains of the identified organisms. The probabilistic methods of steps (b) and (d) may comprise probabilistic matching. The trait-specific reference sequence information contained in the trait-specific database catalog may be a subset of the reference sequence information contained in the reference database.

In some embodiments, the method may include creating a sample sequence library with words or n-mers derived from the unassembled nucleotide fragment reads; and creating a reference sequence library with words or n-mers derived from the reference sequence information. The probabilistic methods may compare the unassembled nucleotide fragment reads with the reference sequence information by comparing words or n-mers from the sample sequence library with words or n-mers from the reference sequence library.

In some embodiments, the method may create a sample sequence library with words or n-mers derived from the unassembled nucleotide fragment reads; and creating a trait-specific sequence library with words or n-mers from the trait-specific reference sequence information. The probabilistic methods may compare the unassembled nucleotide fragment reads with trait-specific reference sequence information contained in the trait-specific database catalog by comparing words or n-mers from the sample sequence library with words or n-mers from the trait-specific sequence library. The trait-specific sequence library may be a library of dictionaries of words from the trait-specific reference sequence information, each dictionary containing words for a particular trait. The sample sequence library may be a sample sequence hash table, and the trait-specific sequence library is a trait-specific hash table.

In some embodiments, the trait-specific reference sequence information contained in the trait-specific database catalog may be closed-genomes, draft genomes, contigs, and/or short reads associated with a particular organism trait. The particular organism trait may be an antibiotic resistance trait, a pathogenicity trait, a bioterror agent marker, or a biochemical trait. Step (c) may comprise scoring and ranking of organism traits likely to be found in the sample.

In some embodiments, the trait-specific reference sequence information contained in the trait-specific database catalog may consist of sequence information of one or more mobile genetic elements. The one or more mobile genetic elements may comprise phages or pathogenicity islands associated with a particular microbial genus or species. Step (c) may determine the probability and relative abundance of the one or more mobile genetic elements.

In some embodiments, the trait-specific reference sequence information contained in the trait-specific database catalog may consist of sequence information associated with a particular phenotypical characteristic. Step (e) may comprise scoring and ranking of particular phenotypical characteristics likely to be found in the sample. The trait-specific reference sequence information contained in the trait-specific database catalog may consist of signature sequences or genome sequences that confirm the presence of particular traits or phenotypes of interest.

In some embodiments, the method may include: (f) performing, by the processing unit, probabilistic matching that compares the unassembled nucleotide fragment reads with second trait-specific reference sequence information contained in a second trait-specific database catalog and produces second probabilistic trait results; and (g) determining, by the processing unit, one or more second traits associated with the organisms using the second probabilistic trait results. The one or more traits may be different than the one or more second traits. The steps (f) and (g) may be performed while steps (b) and (c) are performed.

In some embodiments, the probabilistic methods of step (b) may comprise probabilistic matching. The sample may be a metagenomic sample. The method may include: (d) performing, by the processing unit, probabilistic methods that compare the unassembled nucleotide fragment reads with reference sequence information contained in a reference database containing genomic identities of organisms and produce probabilistic identity results; (e1) for organisms contained in the sample that are contained in the reference database, determining, by the processing unit, the identities of the organisms contained in the sample that are contained in the reference database at least at the species level using the probabilistic identity results; and (e2) for organisms contained in the sample that are not contained in the reference database, determining, by the processing unit, the identities of organisms contained in the reference database that are nearest neighbors to organisms contained in the sample.

In another aspect, the present invention provides an apparatus for characterizing organisms based on sequence information derived from a sample containing genetic material from the organisms. The apparatus may comprise a processing unit including a processor and memory. The processing unit may be configured to: (a) receive the sequence information derived from the sample, wherein the sequence information includes unassembled nucleotide fragment reads; (b) perform probabilistic matching that compares the unassembled nucleotide fragment reads with trait-specific reference sequence information contained in a trait-specific database catalog and produces probabilistic trait results; and (c) determine one or more traits associated with the organisms using the probabilistic trait results.

In some embodiments, the processing unit may be further configured to: (d) perform probabilistic methods that compare the unassembled nucleotide fragment reads with reference sequence information contained in a reference database containing genomic identities of organisms and produce probabilistic identity results; and (e) determine the identities of the organisms at least at the species level using the probabilistic identity results. The processing unit may be further configured to: (f) perform, by the processing unit, probabilistic matching that compares the unassembled nucleotide fragment reads with second trait-specific reference sequence information contained in a second trait-specific database catalog and produces second probabilistic trait results; and (g) determine, by the processing unit, one or more second traits associated with the organisms using the second probabilistic trait results. The one or more traits are different than the one or more second traits.

In some embodiments, the processing unit may be further configured to: create a sample sequence library with words or n-mers derived from the unassembled nucleotide fragment reads; and create a reference sequence library with words or n-mers derived from the reference sequence information. The probabilistic methods may compare the unassembled nucleotide fragment reads with the reference sequence information by comparing words or n-mers from the sample sequence library with words or n-mers from the reference sequence library.

In some embodiments, the processing unit may be further configured to: create a sample sequence library with words or n-mers derived from the unassembled nucleotide fragment reads; and create a trait-specific sequence library with words or n-mers derived from the trait-specific reference sequence information. The probabilistic methods may compare the unassembled nucleotide fragment reads with trait-specific reference sequence information contained in the trait-specific database catalog by comparing words or n-mers from the sample sequence library with words or n-mers from the trait-specific sequence library. The trait-specific sequence library may be a library of dictionaries of words from the trait-specific reference sequence information, each dictionary containing words for a particular trait. The sample sequence library may be a sample sequence hash table, and the trait-specific sequence library is a trait-specific hash table.

In some embodiments, the processing unit may be further configured to: (d) perform probabilistic methods that compare the unassembled nucleotide fragment reads with reference sequence information contained in a reference database containing genomic identities of organisms and produce probabilistic identity results; (e1) for organisms contained in the sample that are contained in the reference database, determine the identities of the organisms contained in the sample that are contained in the reference database at least at the species level using the probabilistic identity results; and (e2) for organisms contained in the sample that are not contained in the reference database, determine the identities of organisms contained in the reference database that are nearest neighbors to organisms contained in the sample.

In yet another aspect, the present invention provides a method of characterizing an organism based on sequence information derived from an isolate containing genetic material from the organism. The method may include: (a) receiving, by a processing unit including a processor and memory, the sequence information derived from the isolate, wherein the sequence information includes unassembled nucleotide fragment reads; (b) performing, by the processing unit, probabilistic matching that compares the unassembled nucleotide fragment reads with trait-specific reference sequence information contained in a trait-specific database catalog and produces probabilistic trait results; and (c) determining, by the processing unit, one or more traits associated with the organism using the probabilistic trait results.

In some embodiments, the method may include: (d) performing, by the processing unit, probabilistic methods that compare the unassembled nucleotide fragment reads with reference sequence information contained in a reference database containing genomic identities of organisms and produce probabilistic identity results; and (e) determining, by the processing unit, the identities of the organism contained in the isolate at least at the species level using the probabilistic identity results. The reference sequence information contained in the reference database may be assembled or partially assembled sequence information. The organism may be a microorganism, and the reference database may comprise a microbial whole genome databases. The method may include determining, by the processing unit, the identity of the organism at the sub-species level using the probabilistic identity results. The method may include determining, by the processing unit, the identity of the organism at the strain level using the probabilistic identity results.

In some embodiments, steps (d) and (e) may be performed while steps (b) and (c) are performed. In other embodiments, steps (b) and (c) may be performed after steps (d) and (e) have been performed.

In some embodiments, the probabilistic methods of steps (b) and (d) may comprise probabilistic matching. The trait-specific reference sequence information contained in the trait-specific database catalog may be a subset of the reference sequence information contained in the reference database.

In some embodiments, the method may include creating a sample sequence library with words or n-mers derived from the unassembled nucleotide fragment reads; and creating a reference sequence library with words or n-mers derived from the reference sequence information. The probabilistic methods may compare the unassembled nucleotide fragment reads with the reference sequence information by comparing words or n-mers from the sample sequence library with words or n-mers from the reference sequence library.

In some embodiments, the method may create a sample sequence library with words or n-mers derived from the unassembled nucleotide fragment reads; and create a trait-specific sequence library with words or n-mers from the trait-specific reference sequence information. The probabilistic methods may compare the unassembled nucleotide fragment reads with trait-specific reference sequence information contained in the trait-specific database catalog by comparing words or n-mers from the sample sequence library with words or n-mers from the trait-specific sequence library. The trait-specific sequence library may be a library of dictionaries of words from the trait-specific reference sequence information, each dictionary containing words for a particular trait. The sample sequence library may be a sample sequence hash table, and the trait-specific sequence library is a trait-specific hash table.

In some embodiments, the trait-specific reference sequence information contained in the trait-specific database catalog may be closed-genomes, draft genomes, contigs, and/or short reads associated with a particular organism trait and/or one or more metagenomic samples. The particular organism trait may be an antibiotic resistance trait, a pathogenicity trait, a bioterror agent marker, or a biochemical trait. The particular organism trait may be a human identity trait, a cancer susceptibility trait, or a disease trait. The trait-specific reference sequence information contained in the trait-specific database catalog may consist of sequence information of one or more mobile genetic elements. The one or more mobile genetic elements may comprise phages or pathogenicity islands associated with a particular microbial genus or species. Step (c) may determine the probability and relative abundance of the one or more mobile genetic elements.

In some embodiments, the trait-specific reference sequence information contained in the trait-specific database catalog may consist of sequence information associated with a particular phenotypical characteristic. Step (e) may comprise scoring and ranking of particular phenotypical characteristics likely to be found in the organism. The trait-specific reference sequence information contained in the trait-specific database catalog may consist of signature sequences or genome sequences that confirm the presence of particular traits or phenotypes of interest.

In some embodiments, the method may include: (f) performing, by the processing unit, probabilistic matching that compares the unassembled nucleotide fragment reads with second trait-specific reference sequence information contained in a second trait-specific database catalog and produces second probabilistic trait results; and (g) determining, by the processing unit, one or more second traits associated with the organism using the second probabilistic trait results. The one or more traits may be different than the one or more second traits. Steps (f) and (g) are performed while steps (b) and (c) are performed.

In some embodiments, the probabilistic methods of step (b) may comprise probabilistic matching. The sample may be a metagenomic sample. The method may include: (d) performing, by the processing unit, probabilistic methods that compare the unassembled nucleotide fragment reads with reference sequence information contained in a reference database containing genomic identities of organisms and produce probabilistic identity results; (e1) if the organism is contained in the reference database, determining, by the processing unit, the identity of the organism at least at the species level using the probabilistic identity results; and (e2) if the organism is not contained in the reference database, determining, by the processing unit, the identity of an organism contained in the reference database that is the nearest neighbor to the organism whose genetic material is contained in the isolate.

In yet another aspect, the present invention provides an apparatus for characterizing an organism based on sequence information derived from an isolate containing genetic material from the organism. The apparatus may comprise a processing unit including a processor and memory. The processing unit may be configured to: (a) receive the sequence information derived from the isolate, wherein the sequence information includes unassembled nucleotide fragment reads; (b) perform probabilistic matching that compares the unassembled nucleotide fragment reads with trait-specific reference sequence information contained in a trait-specific database catalog and produces probabilistic trait results; and (c) determine one or more traits associated with the organism using the probabilistic trait results.

In some embodiments, the processing unit may be further configured to: (d) perform probabilistic methods that compare the unassembled nucleotide fragment reads with reference sequence information contained in a reference database containing genomic identities of organisms and produce probabilistic identity results; and (e) determine the identity of the organism at least at the species level using the probabilistic identity results. The processing unit may be further configured to: (f) perform, by the processing unit, probabilistic matching that compares the unassembled nucleotide fragment reads with second trait-specific reference sequence information contained in a second trait-specific database catalog and produces second probabilistic trait results; and (g) determine, by the processing unit, one or more second traits associated with the organisms using the second probabilistic trait results. The one or more traits may be different than the one or more second traits.

In some embodiments, the processing unit may be further configured to: (d) perform probabilistic methods that compare the unassembled nucleotide fragment reads with reference sequence information contained in a reference database to identify unique sequences along with the occurrence and distribution of non-unique sequences generated from neighboring sequences conserved among other bacteria at different taxonomic levels.

In some embodiments, the unique sequences identified by probabilistic methods are flanked by conserved sequences found in other bacteria to further differentiate one bacterium from another at least at the species level.

In some embodiments, the unique sequences identified by probabilistic methods are capable of being used to design macro or microarrays for identification of microbes at least at the species level.

In some embodiments, the processing unit may be configured to: (d) perform probabilistic methods that compare the unassembled nucleotide fragment reads with reference sequence information contained in a reference database containing genomic identities of organisms and produce probabilistic identity results; (e1) if the organism is contained in the reference database, determine the identity of the organism at least at the species level using the probabilistic identity results; and (e2) if the organism is not contained in the reference database, determine the identity of an organism contained in the reference database that is the phylogenetic nearest neighbor to the organism whose genetic material is contained in the isolate.

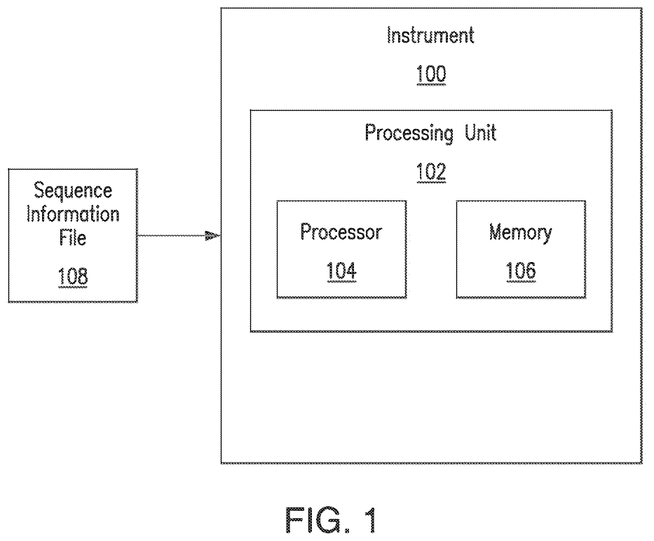

FIG. 1 is a schematic illustration of an instrument 100 according to one embodiment of the present invention. Instrument 100 may be a device capable of characterizing biological material in a sample or isolate. In some embodiments, instrument 100 may be a device capable of characterizing the identities of one or more organisms (e.g., one or more microorganisms, such as bacteria, viruses, parasites, fungi, pathogens, and/or commensals) in a sample or isolate at the species and/or sub-species (e.g., morphovars, serovars, and biovars) level and/or strain level.

Instrument 100 may also be capable of characterizing the relative populations of microorganisms contained in a sample. Instrument 100 may be capable of characterizing one or more traits associated with the biological material contained in a sample or isolate. In some embodiments, the sample may be metagenomic sample. For instance, the metagenomic sample may contain more than one species and/or may contain more than one subspecies within a species. Alternatively or additionally, the metagenomic sample may contain more than multiple genera and can be comprised of bacteria, viruses, and/or fungi.

In some embodiments, instrument 100 may comprise a processing unit 102. The processing unit 102 may include a processor 104 and a memory 106. The processing unit 102 may be configured to perform the characterization of biological material in a sample or isolate. Alternatively, instrument 100 may comprise units in the form of hardware and/or software each configured to perform one or more portions of the characterization of biological material. Further, each of the units may comprise its own processor and memory, or each of the units may share a processor and memory with one or more of the other units.

In some embodiments, instrument 100 may utilize sequence information. The sequence information may be derived from a sample or isolate. In some embodiments, the sample may contain genetic material from a plurality of organisms. In a non-limiting embodiment, the sample may contain a plurality of microbial organisms, including bacteria, viruses, parasites, fungi, plasmids and other exogenous DNA or RNA fragments available in the sample type. In some embodiments, the isolate contains genetic material from one or more organisms that have been isolated from a sample.

In one embodiment, the sequence information may be produced by collecting a sample or isolate containing genetic material, extracting fragments (e.g., nucleic acid and/or protein and/or metabolites) and sequencing the fragments. In some embodiments, the sample is a metagenomic sample, and the extracted and sequenced fragments are metagenomic fragments. In a non-limiting embodiment, the sample may be a subject sample and/or an environment sample. The subject sample (e.g., blood, saliva, etc.) may include the subject's DNA as well as DNA of any organisms (pathogenic or otherwise) in the subject. The environment sample may include, but is not limited to, organisms in their natural state in the environment (including food, air, water, soil, tissue).

In some embodiments, the sequence information may include or be in the form of nucleotide fragment reads. In some embodiments, the sequence information may be unassembled sequence information (i.e., sequence information that has not been assembled into larger contigs or full genomes). For example, in a non-limiting embodiment, the sequence information utilized by the processing unit 102 may include unassembled nucleotide fragment reads.

Instrument 100 may utilize sequence information including hundreds, thousands or millions of short fragment reads (e.g., unassembled fragment reads). The sequence information may be in the form of a sequence information file 108 produced from the fragment reads.

Although fragment reads included in the sequence information and utilized by the processing unit 102 may be greater than 100 base pairs in length, the fragment reads included in the sequence information and utilized by the processing unit 102 may have lengths of approximately 12 to 100 base pairs. For instance, in a non-limiting embodiment, instrument 100 may characterize populations of organisms (e.g., microorganisms) using fragment reads (e.g., metagenomic fragment reads) having lengths of approximately 12 to 15 base pairs, 16 to 25 base pairs, 25 to 50 base pairs or 50 to 100 base pairs. For example, for DNA, the fragment reads may have read lengths of less than 100 base pairs, and the sequence information file 108 produced therefrom may contain millions of DNA fragment reads.

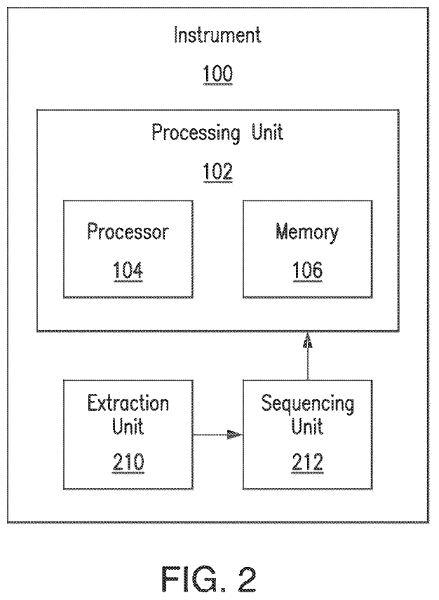

In the embodiment illustrated in FIG. 1, instrument 100 may receive a sequence information file 108 as input. However, in other embodiments, the instrument 100 may receive fragment reads individually and produce a sequence information file 108 including the received fragment reads. In still other embodiments, such as the embodiment illustrated in FIG. 2, instrument 100 may additionally comprise an extraction unit 210 and a sequencing unit 212 and be capable of receiving a sample or isolate as input and producing a sequence information file 108 therefrom. In some embodiments, the extraction unit 210 may extract fragments (e.g., nucleotide fragments) or unamplified single molecules from the sample or isolate and yield a stream of fragments or single molecules. In some embodiments the single molecules may be unamplified single molecules, but, in other embodiments, the extraction unit 210 may use amplification methods.

In some embodiments, the sequencing unit 212 may receive extracted fragments (e.g., nucleotide fragments) or molecules from the extraction unit 210, sequence the received fragments or molecules and producing a sequence information file 108 therefrom. In some embodiments, the sequencing unit 212 may perform sequencing based on, but not limited to, Sequencing-by-synthesis, Sequencing-by-ligation, Single-molecule-sequencing and Pyrosequencing. In one embodiment, the sequencing unit 212 may be interchangeable and removeably coupled to the instrument 100. In a non-limiting embodiment, the sequencing unit 212 may be the interchangeable cassette described in U.S. Patent Application No. 2012/0004111, which is incorporated by reference herein in its entirety.

In some embodiments, instrument 100 may be coupled to an external sequencer and may receive a sequence information file 108 directly from the external sequencer, but this is not required. Instrument 100 may also receive the sequence information file 108 indirectly from one or more external sequencers that are not coupled to instrument 100. For example, instrument 100 may receive a sequence information file 108 over a communication network from a sequencer, which may be located remotely. Or, a sequence information file 108, which has previously been stored on a storage medium, such as a hard disk drive or optical storage medium, may be input into instrument 100.

In addition, instrument 100 may receive a sequence information file 108 or fragments reads in real-time, immediately following sequencing by a sequencer or in parallel with sequencing by a sequencer, but this also is not required. Instrument 100 may also receive a sequence information file 108 or fragments at a later time. In other words, the characterization of biological material in a sample or an isolate performed by instrument 100 may be performed in-line with sample or isolate collection, fragment extraction, and fragment sequencing, but all of the steps may be handled separately and/or in a stepwise fashion.

Instrument 100 may operate under the control of a sequencer that sequences the fragments extracted from a sample or isolate, but no connected processing or even direct communication between instrument 100 and a sequencer is required. Instead, the characterization of biological material in a sample performed by instrument 100 may be performed separately from sample or isolate collection, fragment extraction and/or fragment sequencing.

In some embodiments, the instrument 100 may be a portable handheld electronic device. In non-limiting embodiments, the instrument 100 may include the structure and/or appearance of the portable devices described in U.S. Patent Application Publication No. 2012/0004111, which is incorporated by reference herein in its entirety. However, this is not required. For instance, in other embodiments, instrument 100 may be a computer (e.g., a laptop computer).

In some embodiments, the instrument 100 may be capable of communicating via a communication network. In one embodiment, the communication network may be used to communicate with any potentially relevant entity, such as, for example, First Responder (i.e., Laboratory Response Network, Reference Labs, Seminal Labs, or National Labs), GenBank®, Center for Disease Control (CDC), physicians, public health personnel, medical records, census data, law enforcement, food manufacturers, food distributors, food retailers, and/or any of those described in U.S. Patent Application Publication No. 2012/0004111, which is incorporated by reference herein in its entirety.

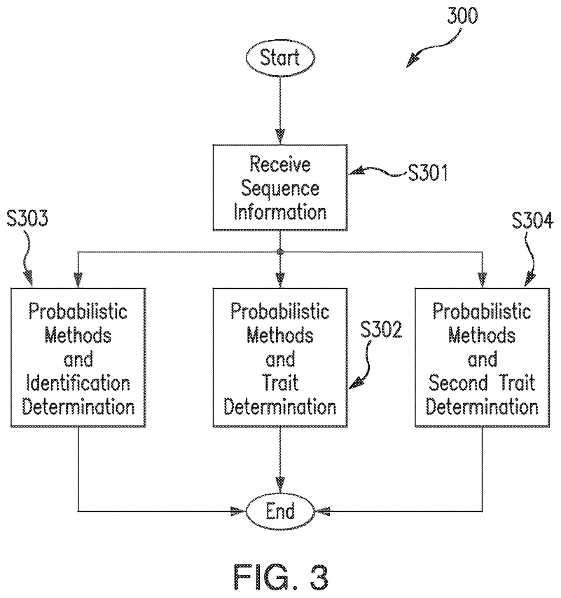

FIG. 3 is flowchart illustrating an embodiment of a process 300 that may be performed to characterize biological material in a sample or isolate. In some embodiments, the steps of process 300 are performed by processing unit 102. In step S301, the instrument 100 and/or processing unit 102 receives sequence information. The sequence information may be in the form of a sequence information file 108. The sequence information may be derived from a sample or isolate containing genetic material from one or more organisms. In some embodiments, the sequence information may include fragment reads. In non-limiting embodiments, the fragment reads may be unassembled fragment reads (e.g., unassembled nucleotide fragment reads). In non-limiting embodiments, the sequence information may have been derived from genetic material contained in a sample or isolate (e.g., fragment reads produced by extracting fragments of the genetic material from the sample or isolate and sequencing the extracted fragments). In some embodiments, the genetic material may be from one or more organisms.

In some embodiments, the process 300 may include one or more steps of probabilistic matching and determination (e.g., steps S302-S304). As shown in the embodiment illustrated in FIG. 3, the process 300 may include a probabilistic method and trait determination step S302. Step S302 may include performing probabilistic methods that produce probabilistic trait results and, using the probabilistic trait results, determining one or more traits (i.e., characteristics) associated with the biological material.

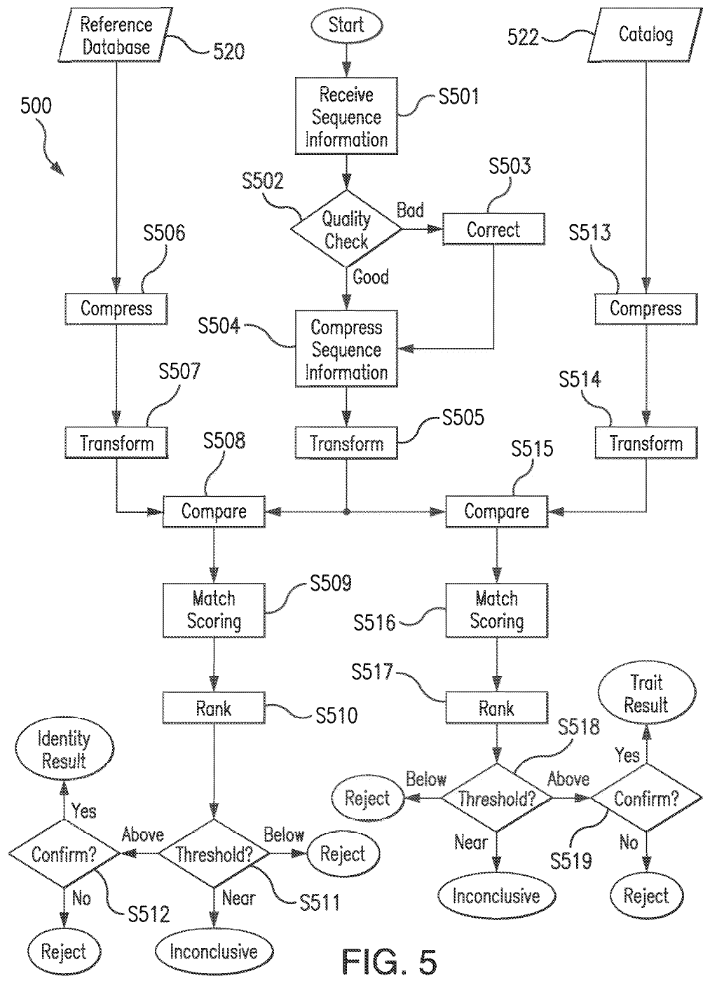

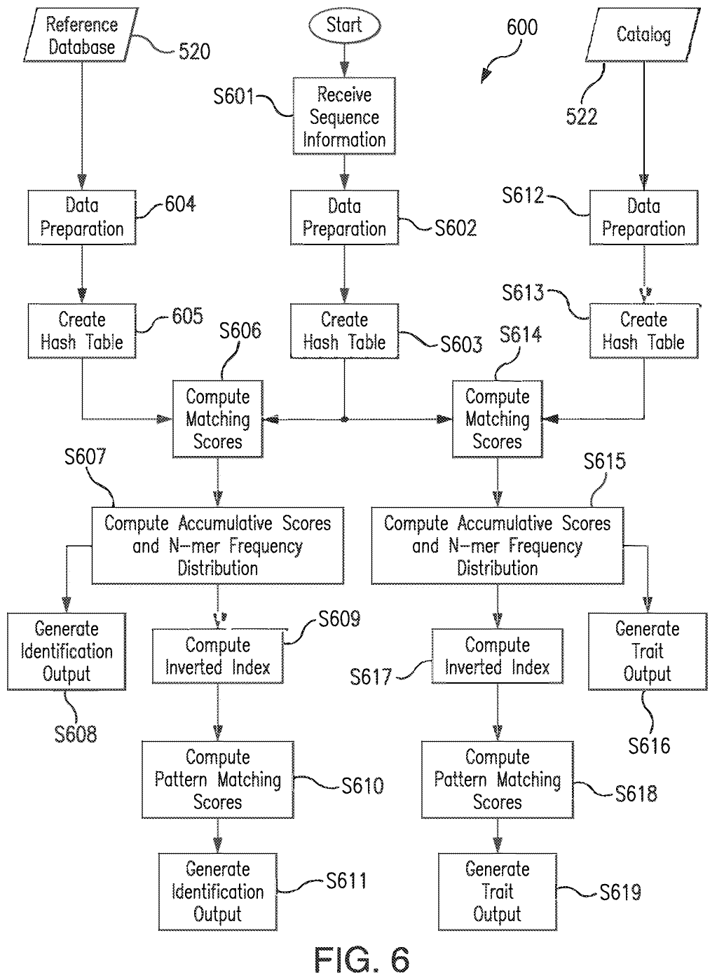

The probabilistic methods performed in step S302 may utilize a trait-specific database catalog (e.g., catalog 522 of FIGS. 5 and 6). The trait-specific database catalog may contain trait-specific reference sequence information (i.e., sequence information contained in the trait-specific database catalog may be associated with one or more particular organism traits). The trait-specific reference sequence information may be, for example, closed-genomes, draft genomes, contigs, and/or short-reads, and each of the closed-genomes, draft genomes, contigs, and/or short-reads may be associated with a particular organism trait. Particular organism traits with which the sequence information contained in the trait-specific database catalog include, but are not limited to, virulence (i.e., fitness) factors, antibiotic resistance traits, pathogenicity traits, bioterror agent markers, biochemical traits, human identity (i.e., ancestry) traits, cancer susceptibility traits, disease traits (e.g., for disease screening), phenotypical characteristics (i.e., phenotypes), mobile genetic elements (i.e., mobilomes such as phages and pathogenicity islands), insertion sequences, transposons, integrons, and/or elements that may be shared generally or restricted to a particular genus, species, or strain. Thus, in some non-limiting embodiments, specific catalogs may be separately maintained to include all sequences involved in mediating (i) drug (antibiotic) resistance, (ii) virulence and pathogenicity, and/or (iii) fitness.

In some embodiments, the sequence information contained in the trait-specific database catalog may be limited to sequence information associated with one or more particular organism traits. Accordingly, the sequence information contained in the trait-specific database catalog may be a subset of the sequence information contained in a reference database (e.g., reference database 520 of FIGS. 5 and 6), which may be a reference genomic database (e.g., GenBank®) containing the genomic identities of organisms.

In some embodiments, the probabilistic methods performed in step S302 may include comparing fragment reads (e.g., unassembled nucleotide fragment reads) included in the received sequence information (e.g., sequence information file 108) with trait-specific reference sequence information contained in the trait-specific database catalog. In some non-limiting embodiments, the probabilistic comparisons performed in the probabilistic methods of step S302 may include, but are not limited to, perfect matching, subsequence uniqueness, pattern matching, multiple sub-sequence matching within n length, inexact matching, seed and extend, distance measurements and phylogenetic tree mapping. In one non-limiting embodiment, the probabilistic methods performed in step S302 may include probabilistic matching.

In some embodiments, the probabilistic methods performed in step S302 may use the Bayesian approach, Recursive Bayesian approach or Naïve Bayesian approach, but the probabilistic methods performed in step S302 are not limited to any of these approaches. In some embodiments, the probabilistic methods performed in step S302 may include scoring and ranking particular organism traits likely to found in the biological material in the sample or isolate.

In some embodiments, step S302 may include determining the probability and relative abundance of one or more particular organism traits in the sample or isolate. For example, in a non-limiting embodiment, step S302 may include determining the probability and relative abundance of one or more mobile genetic elements likely to be found in a sample or isolate. In another non-limiting embodiment, step S302 may include determining the probability and relative abundance of one or more phenotypical characteristic likely to be found in a sample or isolate.

U.S. Patent Application Publication No. 2012/0004111, which is incorporated by reference herein in its entirety, describes probabilistic methods that may be used to characterize the identities and relative populations of organisms in a sample. In some non-limiting embodiments, the probabilistic methods performed in step 302 may be the same as one or more of the probabilistic methods described in U.S. Patent Application Publication No. 2012/0004111 except that the probabilistic methods performed in step 302 compare the received sequence information to trait-specific reference sequence information contained in a trait-specific database catalog as opposed to a reference database containing genomic identities of organisms. As a result, in these non-limiting embodiments, the probabilistic methods performed in step 302 may use the probabilistic methods to characterize (i.e., determine) one or more traits associated with one or more of the organism(s) in the sample or isolate and the relative abundance of the one or more traits associated with one or more of the organism(s) in the sample or isolate (as opposed to characterizing the identities and relative populations of organisms). However, the probabilistic methods performed in step 302 are not limited to those described in U.S. Patent Application Publication No. 2012/0004111 and other probabilistic methods may additionally or alternatively be used.

As shown in the embodiment illustrated in FIG. 3, the process 300 may include a probabilistic method and identification determination step S303. Step S303 may include performing probabilistic methods that produce probabilistic identity results and determining the identities of one or more organisms contained in the sample or isolate. The determination may be based on the probabilistic identity results, and the identities may be determined at least at the species level.

The probabilistic methods performed in step S303 may utilize a reference database (e.g., reference database 520 of FIGS. 5 and 6) containing the genomic identities of organisms. In one non-limiting embodiment, the reference database may be a microbial whole genome database. In another non-limiting embodiment, the reference database may be GenBank®. The reference database may contain reference sequence information. In one embodiment, the reference sequence information may be, for example, assembled or partially assembled sequence information.

In some embodiments, the probabilistic methods performed in step S303 may include comparing fragment reads (e.g., unassembled nucleotide fragment reads) included in the received sequence information (e.g., sequence information file 108) with reference sequence information contained in the reference database. In some non-limiting embodiments, the probabilistic comparisons performed in the probabilistic methods of step S303 may include, but are not limited to, perfect matching, subsequence uniqueness, pattern matching, multiple sub-sequence matching within n length, inexact matching, seed and extend, distance measurements and phylogenetic tree mapping. In one non-limiting embodiment, the probabilistic methods performed in step S303 may include probabilistic matching.

In some embodiments, the probabilistic methods performed in step S303 may use the Bayesian approach, Recursive Bayesian approach or Naïve Bayesian approach, but the probabilistic methods performed in step S303 are not limited to any of these approaches. In some embodiments, the probabilistic methods performed in step S303 may include scoring and ranking organisms likely to found in the biological material in the sample or isolate.

In some embodiments, step S303 may include determining the identities of the organisms contained in the sample at the sub-species level using the probabilistic identity results. In some embodiments, step S303 may include determining the identities of the organisms contained in the sample at the strain level using the probabilistic identity results.

In some embodiments, step S303 may include determining the probability and relative abundance of one or more particular organism traits in the sample or isolate. For example, in a non-limiting embodiment, step S303 may include determining the probability and relative abundance of one or more organisms likely to be found in a sample or isolate. In some embodiments, step S303 may include characterizing (i.e., determining) the relative populations (i.e., concentrations or abundance) of species and/or sub-species and/or strains of the identified organisms.

U.S. Patent Application Publication No. 2012/0004111, which is incorporated by reference herein in its entirety, describes probabilistic methods that may be used to characterize the identities and relative populations of organisms in a sample. In some non-limiting embodiments, the probabilistic methods performed in step 303 may be the same as one or more of the probabilistic methods described in U.S. Patent Application Publication No. 2012/0004111. As a result, in these non-limiting embodiments, the probabilistic methods performed in step 303 may use the probabilistic methods to characterize (i.e., determine) the identities and/or relative populations of organisms in a sample or isolate. However, the probabilistic methods performed in step 303 are not limited to those described in U.S. Patent Application Publication No. 2012/0004111 and other probabilistic methods may additionally or alternatively be used.

In some embodiments, if the sample or isolate contains genetic material from one or more organisms identified in the reference database (i.e., known organisms), step S303 may include determining the identities of the one or more organisms contained in the sample and identified in the reference database. In one embodiment, if the sample or isolate contains genetic material from one or more organisms not identified in the reference database (i.e., unknown organisms), step S303 may include determining the identities of organisms identified in the reference database that are nearest neighbors to the one or more organisms contained in the sample and not identified in the reference database. In this embodiment, the identification of the nearest neighbor may enable location of the one or more organisms contained in the sample and not identified in the reference database within its phylogeny. When applied to an isolate, step S303 may pinpoint the nature of any unknown organisms contained in the isolate (provided that the reference database contains nearest neighbor, assembled whole genomes).

As shown in the embodiment illustrated in FIG. 3, the process 300 may include a probabilistic method and second trait determination step S304. Step S304 may include performing probabilistic methods that produce second probabilistic trait results and, using the second probabilistic trait results, determining one or more second traits (i.e., characteristics) associated with the biological material. Step S304 may correspond to step S302 except that the probabilistic methods performed in step S304 utilize a second trait-specific database catalog instead of the trait-specific database catalog utilized in step S302.

The second trait-specific database catalog utilized in step S304 may contain second trait-specific reference sequence information (i.e., sequence information contained in the second trait-specific database catalog may be associated with one or more second particular organism traits). The one or more second particular traits with which the second trait-specific reference sequence information is associated may be different than the one or more particular traits with which the trait-specific reference sequence information is associated. The second trait-specific reference sequence information may be, for example, closed-genomes, draft genomes, contigs, and/or short-reads, and each of the closed-genomes, draft genomes, contigs, and/or short-reads may be associated with a second particular organism trait.

In some embodiments, the sequence information contained in the second trait-specific database catalog may be limited to sequence information associated with one or more second particular organism traits. Accordingly, the sequence information contained in the trait-specific database catalog may be a subset of the sequence information contained in a reference database (e.g., reference database 520 of FIGS. 5 and 6), which may be a reference genomic database (e.g., GenBank®) containing the genomic identities of organisms.

In some embodiments, the probabilistic methods performed in step S304 may include comparing fragment reads (e.g., unassembled nucleotide fragment reads) included in the received sequence information (e.g., sequence information file 108) with second trait-specific reference sequence information contained in the second trait-specific database catalog. In some non-limiting embodiments, the probabilistic comparisons performed in the probabilistic methods of step S304 may include, but are not limited to, perfect matching, subsequence uniqueness, pattern matching, multiple sub-sequence matching within n length, inexact matching, seed and extend, distance measurements and phylogenetic tree mapping. In one non-limiting embodiment, the probabilistic methods performed in step S304 may include probabilistic matching.

In some embodiments, the probabilistic methods performed in step S304 may use the Bayesian approach, Recursive Bayesian approach or Naïve Bayesian approach, but the probabilistic methods performed in step S304 are not limited to any of these approaches. In some embodiments, the probabilistic methods performed in step S304 may include scoring and ranking second particular organism traits likely to found in the biological material in the sample or isolate.

In some embodiments, step S304 may include determining the probability and relative abundance of one or more second particular organism traits in the sample or isolate. For example, in a non-limiting embodiment, step S304 may include determining the probability and relative abundance of one or more mobile genetic elements likely to be found in a sample or isolate. In another non-limiting embodiment, step S302 may include determining the probability and relative abundance of one or more phenotypical characteristic likely to be found in a sample or isolate.

U.S. Patent Application Publication No. 2012/0004111, which is incorporated by reference herein in its entirety, describes probabilistic methods that may be used to characterize the identities and relative populations of organisms in a sample. In some non-limiting embodiments, the probabilistic methods performed in step 304 may be the same as one or more of the probabilistic methods described in U.S. Patent Application Publication No. 2012/0004111 except that the probabilistic methods performed in step 304 compare the received sequence information to second trait-specific reference sequence information contained in a second trait-specific database catalog as opposed to a reference database containing genomic identities of organisms. As a result, in these non-limiting embodiments, the probabilistic methods performed in step 304 may use the probabilistic methods to characterize (i.e., determine) one or more second traits associated with one or more of the organism(s) in the sample or isolate and the relative abundance of the one or more second traits associated with one or more of the organism(s) in the sample or isolate (as opposed to characterizing the identities and relative populations of organisms). However, the probabilistic methods performed in step 304 are not limited to those described in U.S. Patent Application Publication No. 2012/0004111 and other probabilistic methods may additionally or alternatively be used.

In the embodiment illustrated in FIG. 3, the steps of probabilistic matching and determination (e.g., steps S302-S304) may be performed concurrently (i.e., one or more of the steps of probabilistic matching and determination may be performed while one or more other steps of probabilistic matching and determination are performed). However, this is not required. In other embodiments, one or more of the steps of probabilistic matching and determination may be performed sequentially (i.e., one or more of the steps of probabilistic matching and determination may be performed after one or more other steps of probabilistic matching and determination have been performed performed).

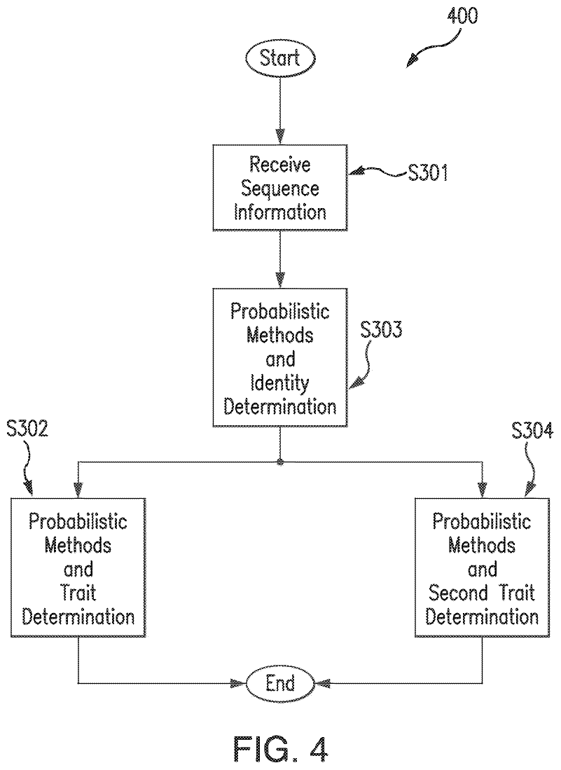

For example, FIG. 4 illustrates an embodiment of a process 400 that may be performed to characterize biological material in a sample or isolate, wherein one or more of the steps of probabilistic matching and determination are performed sequentially. In the embodiment illustrated in FIG. 4, probabilistic methods and trait determination steps (e.g., steps S302 and/or 5304) may be performed after probabilistic methods and identification determination step S303 has been completed.

Although the embodiments of the processes 300 and 400 illustrated FIGS. 3 and 4, respectively, each include two steps of probabilistic methods and trait determination (i.e., steps S302 and S304), this is not required. Some embodiments of the processes 300 and 400 may include one step of probabilistic methods and trait determination (e.g., step S302 and not step S304). Other embodiments of the processes 300 and 400 may include more than two steps of probabilistic methods and trait determination. For example, some embodiments of processes 300 and 400 may have three, four, five, or more steps of probabilistic methods and trait determination with each step of probabilistic methods and trait determination utilizing a different trait-specific database catalog.

Although the processes 300 and 400 for characterizing biological material in a sample or isolate illustrated in FIGS. 3 and 4, respectively, may be performed using a variety of implementations, two particular non-limiting embodiments of comparator engines that may be used to characterize biological material in a sample or isolate are described below with reference to FIGS. 5 and 6, respectively.

The basic premise behind first comparator engine 500 is that the sequence information for an organism can be divided up into words, and that a sub-set of these words can be used to identify the original organism. At a high level, the first comparator engine 500 takes the reference sequence information (e.g., trait-specific reference sequence information contained in a trait-specific reference database catalog and associated with a particular organism trait or reference sequence information contained in a reference genomic database and associated with a genomic identity, such as a particular species or strain), and builds a library of words from the reference sequence information. Then, to analyze sequence information derived from a sample or isolate, the first comparator engine 500 takes the sequence information derived from the sample or isolate and divides that sequence information into a word list. Next, the first comparator engine 500 takes the words from the sequence information derived from the sample or isolate and matches them to the words in the library from the reference sequence information. The matches are then summarized by counting, for each reference sequence, the number of words from the sequence information derived from the sample or isolate that match a word from the reference sequence, which may be, for example, associated with a particular trait or genome identity.

In some embodiments, the steps of the first comparator engine 500 are performed by processing unit 102. In step S501, the first comparator engine 500 may receive sequence information. The sequence information may be in the form of a sequence information file 108. The sequence information may be derived from a sample or isolate containing genetic material from one or more organisms. In some embodiments, the sequence information may include fragment reads. In non-limiting embodiments, the fragment reads may be unassembled fragment reads (e.g., unassembled nucleotide fragment reads).

In step S502, the first comparator engine 500 may perform a quality check of the received sequence information. If the quality of the received sequence information is determined to be good, the first comparator engine 500 may proceed to step S504. However, if the quality of the received sequence information is determined to be bad, the received sequence information may be corrected in step S503 before proceeding to step S504. In some embodiments, the quality check is performed in step S502 because the quality of data may be important for various downstream analyses, such as sequence assembly, single nucleotide polymorphisms identification, gene expression studies as well as microbial identification. Several sequence artifacts, including read errors (base calling errors and small insertions/deletions), poor quality reads and primer/adaptor contaminations are quite common in the NGS data and can impose significant impact on the downstream sequence processing/analysis. The quality check and subsequent correction in step S503 removes these sequence artifacts before downstream analyses to reduce erroneous conclusions. In some embodiments, the quality check may be performed using a quality score assigned to the assigned to the received sequence information software integrated into the sequencing platform(s) (e.g., sequencing unit 212). In one non-limiting embodiment, reads with quality scores of at least Q20 are included, and software to trim the ends of primers is applied.

In step S504, the first comparator engine 500 may compress the received sequence information. In other words, in step S504 the first comparator engine 500 may reduce the data size of the sequence information. For example, the compression step S504 may remove unnecessary information.

In step S505, the first comparator engine 500 may transform the compressed sequence information into an alternative data set (e.g., a list of words from the sequence information). In a non-limiting embodiment, in step S504, the first comparator engine 500 may perform the word finding/parsing process described in U.S. Patent Application Publication No. 2012/0004111, which is incorporated by reference herein in its entirety, with reference to steps S1502 and S1503 of FIG. 15 and FIG. 16.

In step S506, the first comparator engine 500 may compress reference sequence information contained in a reference database 520, which contains genomic identities of organisms. In other words, in step S506 the first comparator engine 500 may reduce the data size of the reference sequence information.

In step S507, the first comparator engine 500 may transform the compressed reference sequence information into an alternative data set (e.g., a library of dictionaries of words from the reference sequence information, each dictionary containing words for a particular genomic identity). In a non-limiting embodiment, in step S507, the first comparator engine 500 may perform the substance cataloging process word finding/parsing process described in U.S. Patent Application Publication No. 2012/0004111, which is incorporated by reference herein in its entirety, with reference to FIGS. 14 and 16.

In step S508, the first comparator engine 500 may compare the words generated in step S505 from the sequence information derived from the sample or isolate to the words generated in step S507 from the reference sequence information. In some embodiments, the comparison may be a many to many comparison. In step S509, the first comparator engine 500 may perform match scoring of the matches identified in step S508. In some embodiments, the first comparator engine 500 may perform match scoring by producing a match scoring table. In some embodiments, the match scoring may include counting, for each organism having reference sequence information in the reference database, the number of words from the sequence information derived from the sample or isolate that match a word from the reference sequence information for the organism. In step S510, the first comparator engine 500 may rank the organisms having reference sequence information in the reference database 520 according to the probability that the organism is contained in the sample or isolate. In a non-limiting embodiment, in steps S508-S510, the first comparator engine 500 may perform the procedures described in paragraphs 0180-0182 of U.S. Patent Application Publication No. 2012/0004111, which is incorporated by reference herein in its entirety, with reference to steps S1504 and S1505 of FIG. 15.

In step S511, the first comparator engine 500 may compare a probability that organisms having reference sequence information in the reference database 520 are in the sample or isolate to a threshold. In some embodiments, if the probability is below the threshold, the first comparator engine 500 may reject the organism. In some embodiments, if the probability is above the threshold, the first comparator engine 500 may accept the organism as contained in the sample or isolate. In one embodiment, if the probability is near the threshold, the first comparator engine 500 may determine that results are inconclusive as to whether the organism is in the sample or isolate.

In some embodiments, the first comparator engine 500 may include a confirming step S512. In step S512, the first comparator engine 500 may optionally confirm or reject the accepted organisms using alternative algorithms. In one embodiment, the confirming step S512 produces an identification result with confidence or probability values for the identification. In some embodiments, the confirming step S512 may additionally or alternatively query a signature database catalog of signature sequences (e.g., nucleic acid signature sequences or genomes). In some embodiments, the confirming step S512 may be optional or may not be included in the first comparator engine 500.

In step S513, the first comparator engine 500 may compress trait-specific reference sequence information contained in a trait-specific database catalog 522. In other words, in step S513 the first comparator engine 500 may reduce the data size of the trait-specific reference sequence information.

In step S514, the first comparator engine 500 may transform the compressed trait-specific reference sequence information into an alternative data set (e.g., a library of dictionaries of words from the trait-specific reference sequence information, each dictionary containing words for a particular trait). In a non-limiting embodiment, in step S514, the first comparator engine 500 may perform the substance cataloging process word finding/parsing process described in U.S. Patent Application Publication No. 2012/0004111, which is incorporated by reference herein in its entirety, with reference to FIGS. 14 and 16 except that a Category or dictionary is created for each trait (as opposed to for each genus, species or strain).

In step S515, the first comparator engine 500 may compare the words generated in step S505 from the sequence information derived from the sample or isolate to the words generated in step S514 from the trait-specific reference sequence information. In some embodiments, the comparison may be a many to many comparison. In step S516, the first comparator engine 500 may perform match scoring of the matches identified in step S508. In some embodiments, the first comparator engine 500 may perform match scoring by producing a match scoring table. In some embodiments, the match scoring may include counting, for each trait having trait-specific reference sequence information in the trait-specific database catalog 522, the number of words from the sequence information derived from the sample or isolate that match a word from the trait-specific reference sequence information for the trait. In step S517, the first comparator engine 500 may rank the traits having trait-specific reference sequence information in the trait-specific database catalog 522 according to the probability that the trait is contained in the sample or isolate.

In a non-limiting embodiment, in steps S515-S517, the first comparator engine 500 may perform the procedures described in paragraphs 0180-0182 of U.S. Patent Application Publication No. 2012/0004111, which is incorporated by reference herein in its entirety, with reference to steps S1504 and S1505 of FIG. 15, except that the matches are to known traits as opposed to known substances (i.e., species or strains of organisms).

In step S518, the first comparator engine 500 may compare a probability that traits having trait-specific reference sequence information in the trait-specific database catalog 522 are in the sample or isolate to a threshold. In some embodiments, if the probability is below the threshold, the first comparator engine 500 may reject the trait. In some embodiments, if the probability is above the threshold, the first comparator engine 500 may accept the trait as contained in the sample or isolate. In one embodiment, if the probability is near the threshold, the first comparator engine 500 may determine that results are inconclusive as to whether the trait is in the sample or isolate.

In some embodiments, the first comparator engine 500 may include a confirming step S519. In step S519, the first comparator engine 500 may optionally confirm or reject the accepted traits using alternative algorithms. In one embodiment, the confirming step S512 produces an identification result with confidence or probability values for the identification. In some embodiments, the confirming step S519 may additionally or alternatively query a signature database catalog of signature sequences (e.g., nucleic acid signature sequences or genomes). In some embodiments, the confirming step S519 may be optional or may not be included in the first comparator engine 500.

In some embodiments, the first comparator engine 500 may perform assembly-free and alignment-free data analysis based on raw reads from DNA sequencers and may build word libraries from reference sequence information in reference genome databases and/or trait-specific database catalogs. In various embodiments, the first comparator engine 500 may be a web-based application tool and may have several password protections. In some embodiments, the first comparator engine 500 may be integrated into a CLC genomics workbench, may manage user accounts with different level of rights, may support fasta, fatsq, and qseq input format, may uploads data files via web browsers and ftp, and/or may allow users to create and update reference databases. In some embodiments, the first comparator engine 500 may allow users to submit multiple jobs, may have proprietary algorithms to process data and create matching scores, may show list of processed experiments, may display ranking scores of genomes identified in the uploaded data file, and/or allow user to sort and filter ranking scores.

In conventional clinical practice of pathogen identification, in general, there are two types of approaches: phenotypic and genotypic. Determining a pathogen by properties of colonies requires waiting for days and is not applicable to non-culturable pathogens.

The genotypic approach may be categorized into three main methods: DNA banding pattern, DNA hybridization, and DNA sequencing. The DNA banding pattern method, depending on successful amplification or/and restriction enzymes, is time and labor consuming, requires high-quality DNA, and lacks reproducibility and resolution to distinguish similar-sized bands. The DNA hybridization-based method (e.g., microarray) suffers cross-hybridization and low reproducibility. The DNA sequencing-based method may sequence only selective genes or partial of genomes and may not be able to differentiate closely related strains even species or entire genomes. The sequencing-based metagenomic approach is a culture-free method to characterize microbes present in samples.

The availability of thousands of sequenced microbial genomes and contemporary high-throughput sequencing technology make identification of pathogens in mixture of genomic sequences in a real-time fashion possible. Conventional metagenomic-based methodologies are generally based on aligning short reads against reference genomes and then clustering the matches or looking for unique features of particular genomes in short reads. Due to their short length, many reads are aligned with more than one reference genomes. The short length of reads may also make finding unique features difficult in short reads. Even when a feature is unique within certain scope, the feature may no longer be unique when the scope is expanded. With these conventional methods, many of those reads are disregarded and not used further. For example, in a human gut analysis published by Qin et al. in 2010, almost half of the data were not utilized because the reads were not found at all or too many were found within reference genome databases. Furthermore, it is a great challenge to process and analyze a massive amount of Next-Gen sequencing data in a short period of time (e.g., within an hour).

FIG. 6 illustrates a non-limiting embodiment of a second comparator engine 600 that may be used to characterize biological material in a sample or isolate. The second comparator engine 600 addresses the abovementioned problems and, in a non-limiting embodiment, is configured to distinguish pathogens even between different strains present in metagenomic data in just a few minutes. In one embodiment, the second comparator engine 600 may take every nucleotide into consideration and may leave no data out. In another embodiment, the second comparator engine 600 may create an n-mer profile and hash the n-mers for each of the available reference genomes (G(i), i˜1 . . . k, where k is number of reference genomes), where n is a user-determined parameter. The n-mer profiles G(i) may be used to interrogate a metagenomic sample or isolate and corresponding distributions S(i) are generated. A threshold value may be computed using a statistical data thresholding method. The second comparator engine 600 may designate all pathogens whose profiling score is above a threshold value as significantly present in a sample or isolate.

In some embodiments, the steps of the second comparator engine 600 are performed by the processing unit 102. In step S601, the second comparator engine 600 may receive sequence information. The sequence information may be in the form of a sequence information file 108. The sequence information may be derived from a sample or isolate containing genetic material from one or more organisms. In some embodiments, the sequence information may include fragment reads. In non-limiting embodiments, the fragment reads may be unassembled fragment reads (e.g., unassembled nucleotide fragment reads).

In step S602, the second comparator engine 600 may prepare the received sequence information. In some embodiments, the second comparator engine 600 may prepare the received sequence information by compressing the received sequence information.

In step S603, the second comparator engine 600 may create a sample or isolate hash table. In some embodiments, the hash table may be created by adding seeds (i.e., tagged n-mers) from each fragment read of the received sequence information. In one embodiment, a seed or tagged n-mer is a sequence (e.g., nucleotide sequence) of n base pairs in length associated with (i.e., adjacent to, following or leading) an anchor, which may be an instance of a particular sequence of m base pairs. In these embodiments, for each instance of the anchor (i.e., the particular sequence of m base pairs) found in the fragment reads from the received sequence information, the seed or tagged n-mer (i.e., the sequence of n base pairs associated with the instance of the particular sequence of m base pairs) is added to the sample or isolate hash table.

In some embodiments, the user may designate the length m of the anchor and/or the length n of the seed or tagged n-mer sequence. In some embodiments, m may be 2 base pairs in length or greater and 8 base pairs in length or shorter. In one embodiment, m may equal 3. In a non-limiting embodiment where m=3, the anchor may be the particular sequence of ATG. In some embodiments, n may be 9 base pairs in length or greater and 20 base pairs in length or shorter. In one embodiment, n may equal 13 base pairs.

In step S604, the second comparator engine 600 may prepare the reference sequence information contained in a reference database 520 containing genomic identities of organisms. In some embodiments (e.g., in embodiments where the data is remote from the processor), the second comparator engine 600 may prepare the reference sequence information by compressing the reference sequence information.

In step S605, the second comparator engine 600 may create a reference hash table. In some embodiments, the reference hash table may be created by adding seeds (i.e., tagged n-mers) from each fragment read of the reference sequence information. In one embodiment, a seed or tagged n-mer is a sequence (e.g., nucleotide sequence) of n base pairs in length associated with (i.e., adjacent to, following or leading) an anchor, which may be an instance of a particular sequence of m base pairs. In these embodiments, for each instance of the anchor (i.e., the particular sequence of m base pairs) found in the fragment reads from the reference sequence information, the seed or tagged n-mer (i.e., the sequence of n base pairs associated with the instance of the particular sequence of m base pairs) is added to the reference hash table.

In step S606, the second comparator engine 600 may compute matching scores between seeds (i.e., tagged n-mers) from the sample or isolate hash table and seeds (i.e., tagged n-mers) of the reference hash table. In some embodiments, the matching scores may be based on an edit distance. In some embodiments, matching begins with the seed and then is extended in both directions until reaching a user-specified threshold value or end of the sequence information.

In step S607, the second comparator engine 600 may compute accumulative scores and an n-mer frequency distribution for each of the organisms in the reference database 520. In step S608, the second comparator engine 600 may generate identification output identifying one or more organisms likely present in the sample or isolate. In some embodiments, the identification output generated in step S608 may be Kepler output.

In step S609, the second comparator engine 600 may create an inverted index of tagged n-mers for specified reference organisms in the reference database 520. In some embodiments, the inverted indexing may be based on pattern aggregation of a subset of above high-scoring genomes and may accomplish further disambiguation. In step S610, the second comparator engine 600 may compute pattern matching scores. In step S611, the second comparator engine 600 may generate additional identification output identifying one or more organisms likely present in the sample or isolate. In some embodiments, the additional identification output generated in step S611 may be Quasar output.

In step S612, the second comparator engine 600 may prepare the trait-specific reference sequence information contained in a trait-specific database catalog 522. In some embodiments, the second comparator engine 600 may prepare the trait-specific reference sequence information by compressing the trait-specific reference sequence information.

In step S613, the second comparator engine 600 may create a trait hash table. In some embodiments, the trait hash table may be created by adding seeds (i.e., tagged n-mers) from each fragment read of the trait-specific reference sequence information. In one embodiment, a seed or tagged n-mer is a sequence (e.g., nucleotide sequence) of n base pairs in length associated with (i.e., adjacent to, following or leading) an anchor, which may be an instance of a particular sequence of m base pairs. In these embodiments, for each instance of the anchor (i.e., the particular sequence of m base pairs) found in the fragment reads from the trait-specific reference sequence information, the seed or tagged n-mer (i.e., the sequence of n base pairs associated with the instance of the particular sequence of m base pairs) is added to the trait hash table.

In step S614, the second comparator engine 600 may compute matching scores between seeds (i.e., tagged n-mers) from the sample or isolate hash table and seeds (i.e., tagged n-mers) of the trait hash table. In some embodiments, the matching scores may be based on an edit distance. In some embodiments, matching begins with the seed and then is extended in both directions until reaching a user-specified threshold value or end of the sequence information.

In step S615, the second comparator engine 600 may compute accumulative scores and an n-mer frequency distribution for each of the traits in the trait-specific database catalog 522. In step S616, the second comparator engine 600 may generate trait output identifying one or more traits likely present in the sample or isolate. In some embodiments, the trait output generated in step S616 may be Kepler output.

In step S617, the second comparator engine 600 may create an inverted index of tagged n-mers for specified reference traits in the trait-specific database catalog 522. In some embodiments, the inverted indexing may be based on pattern aggregation of a subset of above high-scoring traits and may accomplish further disambiguation. In step S618, the second comparator engine 600 may compute pattern matching scores. In step S619, the second comparator engine 600 may generate additional trait output identifying one or more traits of one or more organisms likely present in the sample or isolate. In some embodiments, the additional trait output generated in step S619 may be Quasar output.

In some embodiments, the second comparator engine 600 may compress and store data and be able to process large files (>gigabytes) in a regular laptop. In some embodiments, the second comparator engine 600 may use efficient algorithms to compare data in a fashion of extra high performance. In some embodiments, the second comparator engine 600 may use statistic algorithms to probabilistically filter out significant genomes present in samples.

In some particular embodiments of the present invention, the characterization may be specific to the species and/or sub-species or strain level and may rely on probabilistic matching methods that compare unassembled sequencing information from metagenomic fragment reads to sequencing information of one or more genomic identity databases for identifying and distinguishing bacterial strains.

Some particular embodiments of the present invention relate to systems and methods for the characterization of specific phenotypical characteristics of organisms in a metagenomic sample containing one or a plurality of microorganisms. More particularly, in some particular embodiments, processes similar to those applied to metagenomic analysis of a sample against a reference database catalog containing genomes specific may be applied to a specified characteristic(s) or phenotype(s) and may enable detection, probabilistic ranking and scoring as to whether the specified characteristic(s) or phenotype(s) are present in a sample.

For example, in one embodiment, if the database catalog consists of mobile genomic elements (i.e., mobilomes such as phages and pathogenicity islands associated with a particular microbial genus and species), a process in accordance with embodiments of the invention method may be used to identify the probability and relative abundance of such mobilomes in the metagenomic sample.

Some particular embodiments of the present invention may enable precise determination of microbial populations in a given sample with respect to the specific taxa (e.g., genus, species, sub-species, and/or strain) of bacteria, viruses, parasites, fungi, or nucleic acid fragments including plasmids and mobile genomic components. Some particular embodiments of the present invention may enable simultaneous identification of a plurality of organisms in a given sample with a single test without having any prior knowledge of organisms present in the sample. Some particular embodiments of the present invention may distinguish between very similar or interrelated species, sub-species and strains for medical, agricultural, and industrial applications and also can identify bacteria.

Some particular embodiments of the present invention may rapidly determine background bacterial populations or microbiomes (bacteria), mycobiomes (fungi) and viromes (viruses), at the species and/or subspecies or strain levels. Some particular embodiments of the present invention may diagnose pathogens causing infectious disease or microbial contamination by normalizing results to background populations. Current methods lack the ability to do this. For instance, in food science, such relative comparisons to microbial background, down to the sub-species and/or strain level, may be used to determine the source of food contamination and degree of pathogenicity.

Some particular embodiments of the present invention may produce results in less than 30 minutes. Some particular embodiments of the present invention may utilize nucleic acid fragment sequence data from sequencing machines without the need to first assemble the fragment data into contiguous segments (contigs) or whole genomes.

Some embodiments of the present invention, in cases when a microbial sequence does not exist in the reference database, may identify nearest neighbors and may enable location of the unknown within its phylogeny. For isolates, this may pinpoint the nature of the unknown, provided that the reference database contains nearest-neighbor, assembled whole genomes.

Some embodiments of the present invention may query one or more specific database catalogs of nucleic acid “signature” sequences or genomes to confirm the presence of particular traits or phenotypes of interest, including, but not limited to, antibiotic resistance traits, pathogenicity traits, bioterror agent markers, biochemical traits, etc.

Some embodiments of the present invention may achieve medical diagnostic screening by identifying in a metagenomic sample, concurrently, pathogen populations, virulence (or fitness) factors, and antibiotic resistant determinants, which may be used for the personalized treatment of infectious disease.

Some embodiments of the present invention may be used to screen an isolated sample of unassembled reads for specific phenotypical characteristics and to provide a database catalog containing specific characteristics of clinical interest. These embodiment may be used, for example, in applications such as human identity, cancer screening, and disease screening for specific diseases associated with one or more defined catalogs of genomes.

Some embodiments of the present invention may query one or more specific database catalogs of nucleic acid “signature” sequences or genomes to enhance resolution and sub-species level identification and to distinguish species that have a high degree of overlap of respective genomes.

In some embodiments of the present invention, the probabilistic methods may compare unassembled nucleotide fragment reads with sequences in one or more sequence libraries generated from reference sequence information contained in a reference database to identify unique sequences throughout the genome along with the occurrence and distribution of non-unique sequences generated from neighboring sequences conserved among other bacteria at different taxonomic levels.

In some embodiments of the present invention, the unique sequences identified by probabilistic methods are flanked by conserved sequences found in other bacteria to further differentiate one bacterium from another at least at the species level. For example, in one non-limiting embodiment, the probabilistic methods identify specific sequences from both conserved sequences and its neighborhood (e.g., within a distance of 50-5000 base-pairs of a conserved sequence). In some embodiments, the unique sequences and/or flanked conserved sequences may be unique k-mers and/or words. In a non-limiting embodiment, unique k-mers and/or words may be identified by the processing illustrated in FIGS. 5 and 6. Furthermore, some particular non-limiting embodiments use the unique sequences and the flanked conserved sequences to identify and differentiate closely allied pathotypes of the same species. For example, one such non-limiting embodiment uses the unique sequences and the flanked conserved sequences to distinguish eight strains of Escherichia coli, namely serotypes O157:H7, O104:H4, O26, O45, O103, O111, O121 and O145. For example, identification of unique sequences may enable identification of specific serotypes or pathotypes whereas the distribution of both unique and non-unique sequences may provide one or more serotype specific patterns, which may identify and distinguish each of the pathotypes or serotypes from one another.

Embodiments of the present invention have been fully described above with reference to the drawing figures. Although the invention has been described based upon these preferred embodiments, it would be apparent to those of skill in the art that certain modifications, variations, and alternative constructions could be made to the described embodiments within the spirit and scope of the invention.

For example, although examples focusing on nucleic acid have been provided above, those of skill in the art would understand that the systems and methods of the present invention could be applied to other substances having a sequence nature, such as amino acid sequences in a protein.

What is claimed is:

1. A system for facilitating data processing efficiency and accuracy via anchor-based creation of hash data structures and use thereof, the system comprising:
a computer system comprising one or more processors programmed with computer program instructions that, when executed, cause the computer system to:
obtain an anchor and a seed length range for creating a reference hash data structure derived from reference data;
extract, based on the anchor and the seed length range, reference seeds from the reference data such that each of the extracted reference seeds (i) is a data instance adjacent at least one instance of the anchor in the reference data and (ii) has a length within the seed length range;
hash the extracted reference seeds and create the reference hash data structure with the hashed reference seeds; and
process unassembled sample data using the reference hash data structure and the anchor and the seed length range from the creation of the reference hash data structure to determine characteristics related to the unassembled sample data.

2. The system of claim 1, wherein processing the unassembled sample data comprises:
extracting, based on the anchor and the seed length range, sample seeds from the sample data such that each of the extracted sample seeds (i) is a data instance adjacent at least one instance of the anchor in the sample data and (ii) has a length within the seed length range;
hashing the extracted reference seeds and creating a sample hash data structure with the hashed sample seeds; and
determining the characteristics related to the unassembled sample data based on the sample hash data structure and the reference hash data structure.

3. The system of claim 2, wherein determining the characteristics related to the unassembled sample data comprises:
comparing one or more hashed seeds of the sample hash data structure with one or more hashed seeds of the reference hash data; and
determining the characteristics related to the unassembled sample data based on the comparison indicating matches between hashed seeds associated with the characteristics.

4. The system of claim 3, wherein determining the characteristics related to the unassembled sample data comprises:
determining, based on the comparison, an amount of matches between hashed seeds associated with a first characteristic; and
determining that the first characteristic is a characteristic present in the unassembled sample data based on the amount of matches satisfying a threshold amount.

5. The system of claim 1, wherein the anchor is a sequence of 2 to 8 base pairs in length, and wherein the seed length range is 9 to 20 base pairs in length.

6. A method comprising:
obtaining, by one or more processors, an anchor and a seed length condition for creating a reference data structure;
extracting, by one or more processors, based on the anchor and the seed length condition, reference seeds from the reference data such that each of the extracted reference seeds (i) is a data instance adjacent at least one instance of the anchor in the reference data and (ii) has a length satisfying the seed length condition;
creating, by one or more processors, the reference data structure with the extracted reference seeds; and
processing, by one or more processors, unassembled sample data using the reference data structure and the anchor and the seed length condition from the creation of the reference data structure to determine characteristics related to the unassembled sample data.

7. The method of claim 6, wherein processing the unassembled sample data comprises:
extracting, based on the anchor and the seed length condition, sample seeds from the sample data such that each of the extracted sample seeds (i) is a data instance adjacent at least one instance of the anchor in the sample data and (ii) has a length satisfy the seed length condition;
creating a sample data structure with the extracted sample seeds; and
determining the characteristics related to the unassembled sample data based on the sample data structure and the reference data structure.

8. The method of claim 7, wherein determining the characteristics related to the unassembled sample data comprises:
comparing one or more seeds of the sample data structure with one or more seeds of the reference data; and
determining the characteristics related to the unassembled sample data based on the comparison indicating matches between seeds associated with the characteristics.

9. The method of claim 8, wherein determining the characteristics related to the unassembled sample data comprises:
determining, based on the comparison, an amount of matches between seeds associated with a first characteristic; and
determining that the first characteristic is a characteristic present in the unassembled sample data based on the amount of matches satisfying a threshold amount.

10. The method of claim 6, wherein creating the reference data structure comprises:
hashing the extracted reference seeds; and
creating the reference data structure with the hashed reference seeds.

11. The method of claim 10, wherein processing the unassembled sample data comprises:
extracting, based on the anchor and the seed length condition, sample seeds from the sample data such that each of the extracted sample seeds (i) is a data instance adjacent at least one instance of the anchor in the sample data and (ii) has a length satisfy the seed length condition;
hashing the extracted sample seeds and creating a sample data structure with the extracted sample seeds; and
determining the characteristics related to the unassembled sample data based on the sample data structure and the reference data structure.

12. The method of claim 6, wherein the anchor is a sequence of 2 to 8 base pairs in length, and wherein the seed length condition is 9 to 20 base pairs in length.

13. One or more non-transitory computer-readable storage media comprising instructions that, when executed by one or more processors, cause operations comprising:
obtaining an anchor and a seed length condition for creating a reference data structure;
extracting, based on the anchor and the seed length condition, reference seeds from the reference data such that each of the extracted reference seeds (i) is a data instance adjacent at least one instance of the anchor in the reference data and (ii) has a length satisfying the seed length condition;
creating the reference data structure with the extracted reference seeds; and
processing unassembled sample data using the reference data structure and the anchor and the seed length condition from the creation of the reference data structure to determine characteristics related to the unassembled sample data.

14. The media of claim 13, wherein processing the unassembled sample data comprises:
extracting, based on the anchor and the seed length condition, sample seeds from the sample data such that each of the extracted sample seeds (i) is a data instance adjacent at least one instance of the anchor in the sample data and (ii) has a length satisfy the seed length condition;
creating a sample data structure with the extracted sample seeds; and
determining the characteristics related to the unassembled sample data based on the sample data structure and the reference data structure.

15. The media of claim 14, wherein determining the characteristics related to the unassembled sample data comprises:
comparing one or more seeds of the sample data structure with one or more seeds of the reference data; and
determining the characteristics related to the unassembled sample data based on the comparison indicating matches between seeds associated with the characteristics.

16. The media of claim 15, wherein determining the characteristics related to the unassembled sample data comprises:
determining, based on the comparison, an amount of matches between seeds associated with a first characteristic; and
determining that the first characteristic is a characteristic present in the unassembled sample data based on the amount of matches satisfying a threshold amount.

17. The media of claim 13, wherein creating the reference data structure comprises:
hashing the extracted reference seeds; and
creating the reference data structure with the hashed reference seeds.

18. The media of claim 17, wherein processing the unassembled sample data comprises:
extracting, based on the anchor and the seed length condition, sample seeds from the sample data such that each of the extracted sample seeds (i) is a data instance adjacent at least one instance of the anchor in the sample data and (ii) has a length satisfy the seed length condition;
hashing the extracted sample seeds and creating a sample data structure with the extracted sample seeds; and
determining the characteristics related to the unassembled sample data based on the sample data structure and the reference data structure.

19. The media of claim 13, wherein the anchor is a sequence of 2 to 8 base pairs in length.

20. The media of claim 19, wherein the seed length condition is 9 to 20 base pairs in length.
